(19) United States
(12) Patent Application Publication
(10) Pub. No.: US 2006/0045871 A1
(43) Pub. Date: Mar. 2, 2006

(54) Antioxidant activity from methanotrophic biomass

(76) Inventor: James M. Odom, Kennett Square, PA (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/212,073

(22) Filed: Aug. 25, 2005

Related U.S. Application Data
(60) Provisional application No. 60/607,100, filed on Sep. 2, 2004.

(51) Int. Cl. C12N 1/20; A61K 35/74; C12P 7/66

(52) U.S. Cl. 424/93.4; 424/442; 435/252.3; 435/133

(57) ABSTRACT

A natural antioxidant, Coenzyme Q8, is produced in methanotropic bacteria. However, current processing techniques for methanotrophic biomass leads to the oxidation of the natural antioxidant. The present methods process the dried methanotrophic biomass so that more of the reduced form (i.e. the form having antioxidant activity) is retained. The increased levels of reduced Coenzyme Q8 should increase the shelf life of animal feed produced from the methanotrophic biomass and may alleviate the need to supplement animal feed formations (i.e. fish feed) with additional antioxidants.

This application claims the benefit of U.S. Provisional Application No. 60/607,100, filed Sep. 2, 2004.

### FIELD OF THE INVENTION

This invention is in the field of microbiology. More specifically, this invention pertains to methods to produce Coenzyme Q8 antioxidant compositions derived from methanotrophic biomass for use in animal feeds.

### BACKGROUND OF THE INVENTION

Most bacteria are known to contain representatives of a broad class of isoprenoid compounds known generically as “quinones” or “coenzyme Q” in their cell membranes. The naturally occurring variants of this molecule differ primarily in terms of the length of the isoprenoid side chain (n=number of 5 carbon units; n=8 for Coenzyme Q8).

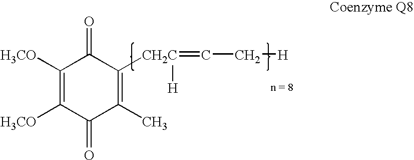

These compounds are thought to function in electron transfer reactions in the cell and they may also offer protection against oxidative damage to the lipid fraction of the cell from reactive oxygen species (Urakami, T., and T. Yoshida, 1993. Journal of Fermentation and Bioengineering, 76:3,191-194 (1993); Takahashi, S. et al., Biochem. Eng. J., 16:183-190 (2003)). Ubiquinones are also known to occur in methanotrophic bacteria (bacteria which utilize only methane) where they function in electron transport form methane to oxygen (Urakami, T., and K. Komagata, J. Gen. Appl. Microbiol., 32:317-341 (1986)).

Antioxidants can be used to stabilize feed materials such as lipid, fat or oil components as well as high value pigments (i.e. carotenoids) from oxidative damage. Pigments, such as astaxanthin and canthaxanthin, are commonly used in fish and poultry feed. Loss of these active pigments due to oxidation is a significant problem. The addition of antioxidants to animal feeds (e.g. fish feed) helps protect and stabilize the feed materials from oxidation, thus increasing the material's quality and shelf life. However, supplementing one or more commercially available antioxidants to animal feed formulations adds significant cost to the final product.

Methanotrophic bacteria biomass can be used as an economical source of protein for animal feed formulations (e.g. “Basic BioProtein”; see WO 01/60974; WO 03/016460; WO 03/068002; WO 03/068003; and WO 03/015534). However, current animal feeds formulations comprised of methanotrophic biomass contain primarily the oxidized form of the natural antioxidants produced by the bacteria. Oxidation is believed to be a result of the current processing and storage methods, where the materials are exposed to oxygen.

The problem to be solved is to provide a method to produce a natural antioxidant (in its reduced form) from a methanotrophic bacterium at levels suitable for use in animal feeds to help protect the compositions from spoilage.

### SUMMARY OF THE INVENTION

The stated problem has been solved by providing methods to produce a natural antioxidant (coenzyme Q8) in its reduced form from methanotrophic biomass. The present method produces the reduced form of coenzyme Q8 (i.e. the active antioxidant) from methanotrophic biomass at levels suitable for use as a natural antioxidant for animal feed formulations, removing the need to supplement the formulations with a separate antioxidant. The ability to produce methanotrophic biomass comprised of one or more reduced antioxidants at high concentrations reduces the cost of producing animal feed compositions and increases the value of the methanotrophic biomass. The present solution reduces problems associated with feed spoilage by providing a low-cast, natural antioxidant in the feed.

Several methanotrophic bacteria (Methylomonas sp. 16a ATCC PTA-2402 and Methylococcus capsulatus NCIMB 11132) have been identified that produce high-levels of coenzyme Q8. However, much of the antioxidant activity is lost using current processing methodology. The present methods have overcome this problem, producing high levels of reduced coenzyme Q8 from methanotrophic biomass for use in animal feed formulations.

Accordingly, the invention provides a methanotrophic biomass composition comprising a coenzyme Q8 in a substantially reduced form.

Additionally, the invention provides a feed composition comprising the methanotrophic biomass of the invention.

In one embodiment, the invention provides a method for the production of a methanotrophic biomass comprising a coenzyme Q8 in substantially reduced form comprising the steps of:
a) providing a methanotrophic biomass comprising coenzyme Q8 in substantially oxidized form;
b) processing the methanotrophic biomass of step (a) under anoxic conditions whereby the coenzyme Q8 is reduced.

Alternatively the invention provides a method for the production of methanotrophic biomass comprising a coenzyme Q8 in substantially reduced form comprising the steps of:
a) providing a methanotrophic bacteria which produces coenzyme Q8 in substantially oxidized form;
b) contacting said methanotrophic bacteria with a growth media comprising:
   1) at least one electron donor species; and
   2) a source of molecular oxygen;
under substantially oxygen free conditions whereby the coenzyme Q8 is substantially reduced to produce a methanotrophic biomass comprising coenzyme Q8 in substantially reduced form; and
c) optionally isolating the methanotrophic biomass of step b).

### BRIEF DESCRIPTION OF THE DRAWINGS AND BIOLOGICAL DEPOSITS

FIG. 1 illustrates the time course of extracted Coenzyme Q8 redox state.

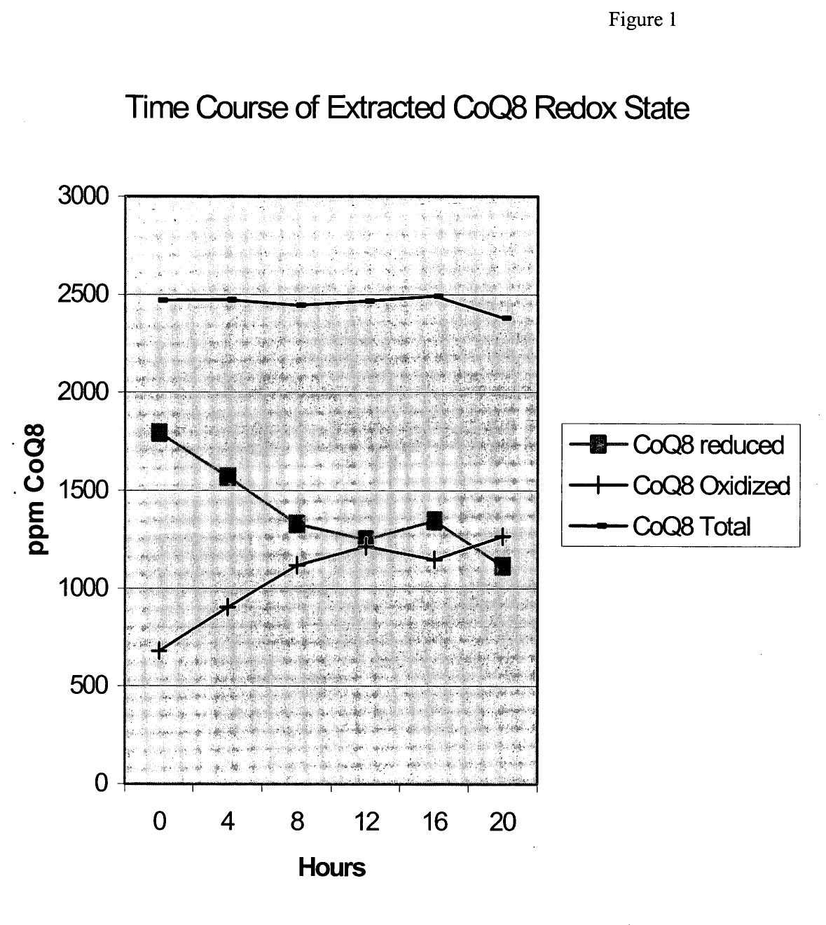

The following biological deposit has been made under the terms of the Budapest Treaty on the International Recognition of the Deposit of Microorganisms for the Purposes of Patent Procedure:

| | International | |
|---|---|---|
| Depositor Identification | Depository | |
| Reference | Designation | Date of Deposit |
| Methylomonas 16a | ATCC PTA 2402 | Aug. 22, 2000 |

As used herein, “ATCC” refers to the American Type Culture Collection International Depository Authority located at ATCC, 10801 University Blvd., Manassas, Va. 20110-2209, U.S.A. The “International Depository Designation” is the accession number to the culture on deposit with ATCC.

The listed deposit will be maintained in the indicated international depository for at least thirty (30) years and will be made available to the public upon the grant of a patent disclosing it. The availability of a deposit does not constitute a license to practice the subject invention in derogation of patent rights granted by government action.

### DETAILED DESCRIPTION

The present invention provides a methanotrophic biomass comprising a significant level of Co-Q8 in substantially reduced form. The biomass produced from these organisms can be processed using the present methods to produce a value-added antioxidant product that eliminates and/or reduces the need=to supplement animal feeds with additional antioxidants.

The high-levels of coenzyme Q8 produced by the biomass of the present invention are particularly useful where all of the Co-Q8 is present in the reduced form. Additionally, the Co-Q8 antioxidant needs little further processing or purification when used in a feed composition. Methanotrophic biomass where Co-C8 are at levels of only 10% by weight, result in final concentration of coenzyme Q8 exceeding 100 ppm in the feed, providing a very effective level of protecting antioxidant. Additionally it is well-known that coenzyme Q compounds are insoluble in water and therefore, within the cell, they partition into the cell membrane, simplifying purification.

In this disclosure, a number of terms and abbreviations are used. The following definitions are provided.

As used herein, “coenzyme Q” or “Co-Q” and “ubiquinone” will be used interchangeably and will refer lipophillic redox-active molecules. In its reduced state, coenzyme Q acts as an antioxidant.

As used herein, the term “NCIMB” refers to the National Collections of Industrial Food and Marine Bacteria, Aberdeen, Scotland.

As used herein, the term “methanotroph” or “methanotrophic bacteria” means a prokaryote capable of utilizing methane as its primary source of carbon and energy. Complete oxidation of methane to carbon dioxide occurs by aerobic degradation pathways. Typical examples of methanotrophs useful in the present invention include (but are not limited to) the genera Methylomonas, Methylobacter, Methylococcus, and Methylosinus. Exemplified in the present examples are Methylococcus capsulatus (Bath) (NCIMB 11132) and Methylomonas sp. 16a (ATCC PTA-2402).

As used herein, the term “high growth methanotrophic bacterial strain” refers to a bacterium capable of growth with methane or methanol as the sole carbon and energy source and which possesses a functional Embden-Meyerof carbon flux pathway resulting in a high rate of growth and yield of cell mass per gram of C₁ substrate metabolized. The specific “high growth methanotrophic bacterial strain” described herein is referred to as “Methylomonas 16a”, “16a” or “Methylomonas sp. 16a”, which terms are used interchangeably and which refer to the Methylomonas sp. 16a (ATCC PTA-2402; U.S. Pat. No. 6,689,601).

As used herein, the term “methanotrophic biomass” is used to describe the biomass produced from methanotrophic bacteria (WO 01/60974). The “biomass material” includes whole cell material and derivatives thereof (e.g. a homogenates, autolysates, or hydrolysates) as well as purified or partially-purified forms of coenzyme Q8 isolated from the methanotrophic biomass.

As used herein, “oxygen free”, “anoxic” or “in the absence of oxygen” are used to describe a condition where oxygen is not present (or is present is very low concentrations) to limit the rate of coenzyme Q8 oxidation. In order to protect the reduced coenzyme Q8 from oxidation, “oxygen free” conditions are used to process and store the methanotrophic biomass and/or the animal feed formulations comprised of methanotrophic biomass. In one embodiment, “substantially oxygen free” means processing conditions comprising less than about 10% molecular oxygen, preferably less than about 5%, more preferable less than about 1%, even more preferably less than about 0.5%, and most preferably less than about 0.1%.

As used herein, the term “C₁ carbon substrate” refers to any carbon-containing molecule that lacks a carbon-carbon bond. Non-limiting examples are methane, methanol, formaldehyde, formic acid, formate, methylated amines (e.g., mono-, di-, and tri-methyl amine), methylated thiols, and carbon dioxide. In one preferred embodiment, the C₁ carbon substrate is methane and/or methanol.

As used herein, the term “C₁ metabolizer” refers to a microorganism that has the ability to use a single carbon substrate as its sole source of energy and biomass. C₁ metabolizers will typically be methylotrophs and/or methanotrophs. The term “C₁ metabolizing bacteria” refers to bacteria that have the ability to use a single carbon substrate as their sole source of energy and biomass. C₁ metabolizing bacteria, a subset of C₁ metabolizers, will typically be methylotrophs and/or methanotrophs. In one preferred aspect, C₁ metabolizers of the present invention are methanotrophic bacteria grown on methane and/or methanol. In another aspect, the methanotroph bacteria are high-growth methanotrophic bacteria grown on methane and/or methanol.

As used herein, “substantially reduced form of coenzyme Q8” or “substantially reduced” refers a condition where the majority of the coenzyme Q8 present in a composition is in the reduced form. In one aspect, substantially reduced refers to a condition where at least 50% of the coenzyme Q8 (percentage based on total coenzyme Q8 present) in a composition is in the reduced state, preferably at least 70% in the reduced form, more preferably at least 80% in the reduced form, even more preferably at least 90% in the reduced form, yet even more preferably at least 95% in the reduced form, and most preferably at least 98% in the reduced form. In another embodiment, the concentration of substantially reduced coenzyme Q8 in the methanotrophic biomass is at least 250 ppm, preferably at least 500, more preferably at least 1000 ppm, even more preferably at least 1500 ppm, and most preferably at least 2000 ppm

The Methanotrophic Biomass

Methanotrophic biomass of the invention comprising Co-Q8, can be generated by growth of the methanotrophic microbes on methane and/or methanol, air, and minerals. Biomass derived from this growth may be in the form of whole microbial cells, disrupted cells or fractions of the disrupted biomass. Bacterial cells suitable for production of methanotrophic biomass include, but are not limited to those belonging to the genera Methylomonas, Methylobacter, Methylococcus, and Methylosinus. This biomass is chemically comprised of all the components of the cell such as protein, lipid, carbohydrate and small molecules. The biomass may be viewed as a carrier for the antioxidant, but will also have intrinsic nutritional value as a source of protein and energy for metabolism of animals fed the material.

Methods to process the methanotrophic biomass have been previously described (WO 01/60974; EP1416808; GB 2385767; WO 03/68002; and WO 03/68003). The methanotrophic biomass produced during fermentation may be isolated using well-known techniques such as centrifugation and/or filtration (i.e. ultrafiltration). Typically, homogenized and non-homogenized materials are subjected to dewatering (e.g. spray drying) and optional sterilization prior to use as a feed material or feed additive. A process to commercially grow methanotropic bacteria using oxygen and a C₁ carbon source together with a nutrient mineral solution in a tubular reactor has been previously described (WO 01/60974). In the present invention, suitable growth conditions are used to grow and produce the methanotrophic biomass. The biomass produced, or a derivative thereof, is then subjected to oxygen free (or at least to very low oxygen concentrations) conditions for a period of time sufficient to fully reduce the coenzyme Q8 and to protect the reduced form of coenzyme Q8. Subsequent processing techniques are conducted under oxygen free conditions. The processing environment can be controlled to limit/prevent exposure of the reduced coenzyme Q8 by processing the methanotrophic biomass under a blanket of a suitable gas, such as nitrogen or carbon dioxide.

The biomass of the invention contains coenzyme Q8 at a concentrations of at least about 1000 ppm, and more specifically in the range of about 1000 ppm to about 2500 ppm (mg total coenzyme Q8/kg dry weight of biomass). This total content of coenzyme Q8 is the combined amount of oxidized and reduced form. The reduced form the coenzyme Q is the only form that has activity as an antioxidant in the absence of any other source of electrons or reducing power. The relative ratio of reduced/oxidized forms will depend on a number of factors including: 1) the ratio of methane to oxygen at the time the biomass is removed from the liquid fermentation, 2) the amount of aeration and exposure to oxygen the biomass receives subsequent to removal from the fermentation liquid but prior to storage, and 3) the storage conditions such as temperature, age and exposure to oxygen of the biomass after it has been stored.

Industrial Production

Industrial production of methanotrophic biomass is well know in the art, see for example (EP 1419234; WO 01/60974; and WO 03/16460), and large-scale production of a product from a microbial host may be produced by a variety of techniques including both batch and continuous culture methodologies.

A classical batch culturing method is a closed system where the composition of the media is set at the beginning of the culture and not subject to artificial alterations during the culturing process. Thus, at the beginning of the culturing process the media is inoculated with the desired organism or organisms and growth or metabolic activity is permitted to occur adding nothing to the system. Typically, however, a “batch” culture is batch with respect to the addition of carbon source and attempts are often made at controlling factors such as pH and oxygen concentration. In batch systems the metabolite and biomass compositions of the system change constantly up to the time the culture is terminated. Within batch cultures cells moderate through a static lag phase to a high growth log phase and finally to a stationary phase where growth rate is diminished or halted. If untreated, cells in the stationary phase will eventually die. Cells in log phase are often responsible for the bulk of production of end product or intermediate in some systems. Stationary or post-exponential phase production can be obtained in other systems.

A variation on the standard batch system is the fed-batch system. Fed-batch culture processes are also suitable in the present invention and comprise a typical batch system with the exception that the substrate is added in increments as the culture progresses. Fed-batch systems are useful when catabolite repression is apt to inhibit the metabolism of the cells and where it is desirable to have limited amounts of substrate in the media. Measurement of the actual substrate concentration in fed-batch systems is difficult and is therefore estimated on the basis of the changes of measurable factors such as pH, dissolved oxygen and the partial pressure of waste gases such as CO₂. Batch and fed-batch culturing methods are common and well known in the art and examples may be found in Biotechnology: A Textbook of Industrial Microbiology, Thomas D. Brock, Second Edition (1989) Sinauer Associates, Inc., Sunderland, Mass., (hereinafter “Brock”) or by Deshpande, Mukund V., Appl. Biochem. Biotechnol., 36:227 (1992) (hereinafter “Deshpande”).

Commercial production of methanotrophic biomass comprised of coenzyme Q8 may also be accomplished with a continuous culture. Continuous cultures are an open system where a defined culture media is added continuously to a bioreactor and an equal amount of conditioned media is removed simultaneously for processing. Continuous cultures generally maintain the cells at a constant high liquid phase density where cells are primarily in log phase growth. Alternatively, continuous culture may be practiced with immobilized cells where carbon and nutrients are continuously added, and valuable products, by-products or waste products are continuously removed from the cell mass. Cell immobilization may be performed using a wide range of solid supports composed of natural and/or synthetic materials.

Continuous or semi-continuous culture allows for the modulation of one factor or any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by media turbidity, is kept constant. Continuous systems strive to maintain steady state growth conditions and thus the cell loss due to media being drawn off must be balanced against the cell growth rate in the culture. Methods of modulating nutrients and growth factors for continuous culture processes as well as techniques for maximizing the rate of product formation are well known in the art of industrial microbiology and a variety of methods are detailed by Brock, supra.

Oxygen Free Processing and Storage of the Methanotrophic Biomass

Methanotrophic bacteria are typically grown under aerobic conditions in the presence of methane (and/or methanol) and nutrient minerals. Methanotrophs convert the carbon source into a variety of complex molecules, including coenzyme Q8. For the production of biomass methanotrophic bacteria are grown to a suitable cell densities and then subsequently harvested to collect the biomass. Typically the biomass is harvested and stored in the presence of oxygen, leading to the oxidation of any endogenous coenzyme Q8, and decreasing its desired antioxidant activity. The process of the present invention avoids this effect by producing and processing the biomass in anoxic conditions. Accordingly the invention provides a method for the production of a methanotrophic biomass comprising a coenzyme Q8 in substantially reduced form comprising the steps of:
a) providing a methanotrophic biomass comprising coenzyme Q8 in substantially oxidized form; and
b) processing the methanotrophic biomass of step (a) under anoxic conditions whereby the coenzyme Q8 is reduced.

More specifically the process of the invention allows for the anoxic growth of methanotrophic bacteria in the presence of at least one electron donor and in the presence of a source of molecular oxygen. Typically the electron donor may also be the carbon substrate, i.e. methanol or methane. The concentrations of the electron donor and the molecular oxygen may be varied to give the maximum reducing effect on the CO-Q8. Thus it is within the context of the present invention to provide a method for the production of methanotrophic biomass comprising a coenzyme Q8 in substantially reduced form comprising the steps of:
a) providing a methanotrophic bacteria which produces coenzyme Q8 in substantially oxidized form;
b) contacting said methanotrophic bacteria with a growth media comprising:
   1) at least one electron donor species; and
   2) a source of molecular oxygen;
under substantially oxygen free conditions whereby the coenzyme Q8 is substantially reduced to produce a methanotrophic biomass comprising coenzyme Q8 in substantially reduced form; and
d) optionally isolating the methanotrophic biomass of step b).

In one aspect, the fermentor conditions (upon achieving a desired cell density) are altered so that oxygen is removed from the fermentor for a period of time to more fully reduce the total amount of coenzyme Q8 in the cells. The suitable amount “oxygen free” time necessary to produce suitable amounts of reduced coenzyme Q8 can be determined by the skilled person by analyzing the methanotrophic biomass and/or fermentor composition. In one aspect, the oxygen-free period is at least 5 minutes, preferably at least about 30 minutes, more preferably at least about 1-hour. In another embodiment, the methanotrophic biomass produced in the fermentor may be transferred to one or more different vessels/tanks where the oxygen free period occurs.

In a preferred process, all subsequent processing steps (i.e. filtration, centrifugation, homogenization, autolysis, hydrolysis, dewatering, etc.) used to produce and store the methanotrophic biomass and/or the purified or partially purified reduced coenzyme Q8 occur under anoxic process conditions. Non-reactive gases (i.e. nitrogen, carbon dioxide, etc.) can be used to blanket the materials to protect the reduced coenzyme Q8 from oxidation. Given the levels of coenzyme Q8 produced by methanotrophic bacteria and the present processing conditions, the methanotrophic biomass will contain a natural antioxidant at levels suitable to protect animal-feed products from spoilage, reducing or eliminating the need to add a commercially available antioxidant to feed formulation.

In an alternative embodiment, it is possible to treat the methanotrophic biomass and/or the purified or partially-purified coenzyme Q8 with a reducing agent to convert oxidized coenzyme Q8 to its reduced form. This additional embodiment can be used to maximize the amount of antioxidant (i.e. the reduced form) produced by the methanotrophic biomass. Suitable reducing agents are known in the art and can be chosen based on cost, availability, of effectiveness. Non-limiting examples of reducing agents can include, sugars, pyruvate, sodium hydrosulfite, sodium borohydride, and ascorbic acid; to name a few.

In another embodiment, the methanotrophic biomass comprised of reduced coenzyme Q8 or the purified or partially-purified coenzyme Q8 produced from methanotrophic biomass is stored in containers under anoxic conditions prior to being incorporated into an animal feed formulation. In a further embodiment, the material comprised of reduced coenzyme Q8 is stored under an oxygen free gas blanket (i.e. under a nitrogen blanket). In yet a further embodiment, the animal feed formulated from the methanotrophic biomass or an animal feed formulated using purified or partially-purified reduced coenzyme Q8 isolated from methanotrophic biomass is stored under anoxic conditions (for example, under an nitrogen blanket) to reduce spoilage and extend the shelf life of the feed material.

In another embodiment, the methanotrophic biomass used to create animal feed formulations contains a sufficient amount of reduced coenzyme Q8 to eliminate the need to supplement the feed with additional antioxidants.

The methanotrophic biomass comprised of the reduced coenzyme Q8 can be used in an animal feed formulation. In another embodiment, the animal feed is a poultry, fish, or shellfish feed.

In another aspect, the methanotrophic biomass comprised of the reduced coenzyme Q8 can be used in other commercial applications such personal care products and cosmetics, to name a few.

In a further aspect, the coenzyme Q8 produced by the methanotrophic bacteria can be purified or partially purified prior to incorporation in one or more of the above compositions.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

### General Methods

The meaning of abbreviations is as follows: “h” means hour(s), “min” means minute(s), “seq” means second(s), “d” means day(s), “mL” means milliliters, “μL” mean microliters, “L” means liters, “g” means grams, “mg” means milligrams, “μg” means micrograms, “avg” means average value, “MW” means molecular weight, “conc” means concentration, “mM” means millimolar, “M” means molar, and “ppm” means parts per million.

Cell Cultivation.

Materials and methods suitable for the maintenance and growth of bacterial cultures are well known in the art. Techniques suitable for use in the following examples may be found as set out in Manual of Methods for General Bacteriology (Phillipp Gerhardt, R. G. E. Murray, Ralph N. Costilow, Eugene W. Nester, Willis A. Wood, Noel R. Krieg and G. Briggs Phillips, eds), American Society for Microbiology, Washington, D.C. (1994)), or by Brock, supra or Deshpande, supra. All reagents and materials used for the growth and maintenance of bacterial cells were obtained from Aldrich Chemicals (Milwaukee, Wis.), DIFCO Laboratories/BD Diagnostics (Sparks, Md.), Promega (Madison, Wis.), New England Biolabs (Beverly, Mass.), GIBCO/BRL Life Technologies (Carlsbad, Calif.), or Sigma Chemical Company (St. Louis, Mo.) unless otherwise specified.

Cells of Methylococcus capsulatus NCIMB 11132 or Methylomonas 16a (ATCC PTA-2402) were cultivated on a defined medium termed “BTZ-3” which comprised only water, mineral salts and a vitamin mixture.

“BTZ-3” Nitrate Medium

Nitrate liquid medium, also referred to herein as “defined medium” or “BTZ-3” medium is comprised of various salts mixed with Solution 1 as indicated below (Tables 1 and 2) or where specified the nitrate is replaced with 15 mM ammonium chloride. Solution 1 provides the composition for 100-fold concentrated stock solution of trace minerals.

TABLE 1
Solution 1*

| | | Conc. | |
|---|---|---|---|
| | MW | (mM) | g per L |
| Nitriloacetic acid | 191.1 | 66.9 | 12.8 |
| CuCl₂ × 2H₂O | 170.48 | 0.15 | 0.0254 |
| FeCl₂ × 4H₂O | 198.81 | 1.5 | 0.3 |
| MnCl₂ × 4H₂O | 197.91 | 0.5 | 0.1 |
| CoCl₂ × 6H₂O | 237.9 | 1.31 | 0.312 |
| ZnCl₂ | 136.29 | 0.73 | 0.1 |
| H₃BO₃ | 61.83 | 0.16 | 0.01 |
| Na₂MoO₄ × 2H₂O | 241.95 | 0.04 | 0.01 |
| NiCl₂ × 6H₂O | 237.7 | 0.77 | 0.184 |

*Mix the gram amounts designated above in 900 mL of H₂O, adjust to pH = 7, and add H₂O to an end volume of 1 L. Keep refrigerated.

TABLE 2
Nitrate liquid medium (BTZ-3)**

| | | Conc. | | |
|---|---|---|---|---|
| | MW | (mM) | g per L | |
| NaNO₃ | 84.99 | 10 | 0.85 | |
| KH₂PO₄ | 136.09 | 3.67 | 0.5 | |
| Na₂SO₄ | 142.04 | 3.52 | 0.5 | |
| MgCl₂ × 6H₂O | 203.3 | 0.98 | 0.2 | |
| CaCl₂ × 2H₂O | 147.02 | 0.68 | 0.1 | |
| 1 M HEPES (pH 7) | 238.3 | | 50 | mL |
| Solution 1 | | | 10 | mL |

**Dissolve in 900 mL H₂O. Adjust to pH = 7, and add H₂O to give 1 L.
For agar plates: Add 15 g of agarose in 1 L of medium, autoclave, let cool down to 50° C., mix, and pour plates.

The cultures were grown under 25% methane in air in a liquid volume of 100 mL within a serum capped 500-mL bottle.

### Coenzyme Q8 Extraction

Lyophilized biomass (26 mg) was mixed with 1 mL of 50:50 methanol:tetrahydrofuran. The mixture was shaken for 15 min and then passed through a 0.2-micron filter before loading onto the HPLC.

Samples were analyzed using the HPLC method described by Crane, F. L. and Barr, R. (Methods Enzymol., 18C:137-165 (1971)).

### Example 1

### Determination of Coenzyme Q8 Content in Methylomonas sp. 16a and Methylococcus capsulatus

Coenzyme Q8 content in Methylomonas 16a (ATCC PTA-2402) biomass and the Norferm “Bioprotein” dried powder product (see WO 01/60974 and GB 0203307.4). Methylomonas sp. 16a was grown to stationary phase in bottles and then harvested by centrifugation (10,000 rpm for 20 min in a SS34 Sorvall rotor). The pelleted cells were then lyophilized under a vacuum for 24 h to remove any traces of water. The analyses show that both the Norferm Product, the freshly grown Methylomonas and M. capsulatus contain coenzyme Q8. However, the Norferm product and the freshly grown M. capsulatus were relatively oxidized compared to the freshly grown Methylomonas material (Table 3).

TABLE 3
Oxidized and reduced forms of coenzyme Q8 measured in
several bacterial samples

| | Avg. | Avg. | Total |
|---|---|---|---|
| | Coenzyme Q8, | Coenzyme Q8 | reduced + oxidized |
| | (reduced) | (oxidized) | Coenzyme Q, |
| Sample | (ppm) | (ppm) | (ppm) |
| Norferm | 0 | 1200 | 1200 |
| “Bioprotein” | | | |
| M. capsulatus | 0 | 1330 | 1330 |
| Methylomonas sp. | 1640 | 990 | 2630 |
| 16a wild type | | | |

### Example 2

### Oxidation Time Course of the Reduced Form of Coenzyme Q8

Time course of oxidation of the reduced form of Coenzyme Q8 after the coenzyme has been extracted from the cells into the methanol/THF solvent. Individual samples of biomass (from Methylomonas sp. 16a) of approximately 27 mg each were extracted into the solvent and then left exposed to the air for up to 19 h (Table 4). The data show the increase in the amount of the oxidized form of Coenzyme Q8 over time, corresponding to a decrease in the amount of the reduced form. Note that the total amount (oxidized+reduced) remains relatively constant. This data demonstrates the oxygen-reactive nature of the reduced form of coenzyme Q8 as extracted from Methylomonas.

TABLE 4
Time course of oxidation for coenzyme Q8

| | Concen- | Coenzyme Q8, | Coenzyme Q8 | Total^{a} |
|---|---|---|---|---|
| | tration | (reduced) | (oxidized) | Coenzyme Q8 |
| Sample | mg/mL | (ppm) | (ppm) | (ppm) |
| sp16a | 27.2 | 1804 | 663 | 2467 |
| sp16a | 27.2 | 1783 | 695 | 2478 |
| sp16a-1 | 24.4 | 1593 | 882 | 2475 |
| sp16a-1 | 24.4 | 1565 | 915 | 2481 |
| sp16a-1 | 24.4 | 1549 | 919 | 2468 |
| sp16a-2 | 24.9 | 1329 | 1097 | 2426 |
| sp16a-2 | 24.9 | 1339 | 1120 | 2460 |
| sp16a-2 | 24.9 | 1314 | 1138 | 2451 |
| sp16a-3 | 24.7 | 1282 | 1214 | 2496 |
| sp16a-3 | 24.7 | 1249 | 1205 | 2455 |
| sp16a-3 | 24.7 | 1224 | 1230 | 2454 |
| sp16a-4 | 26.7 | 1349 | 1141 | 2490 |
| sp16a-4 | 26.7 | 1342 | 1157 | 2500 |
| sp16a-4 | 26.7 | 1345 | 1145 | 2490 |
| sp16a-5 | 25.1 | 1107 | 1274 | 2381 |
| sp16a-5 | 25.1 | 1117 | 1265 | 2382 |
| sp16a-5 | 25.1 | 1122 | 1261 | 2383 |

^{a}= Oxidized and reduced forms of coenzyme Q8 combined.

### Example 3

### Process for Obtaining Coenzyme Q from Methylomonas in Reduced Form

The objective of Example 3 is to illustrate process conditions that will produce the maximum amount of Coenzyme Q8 in its reduced state. The reduced form is capable of reaction with molecular oxygen such that molecular oxygen is removed.

Methylomonas (or any methanotrophic bacterium) requires a C₁ carbon source (methane and/or methanol), air, and minerals for growth. These can be provided in a controlled manner by continuous culture in a gas-sparged vessel where mineral nutrients, and gaseous substrates are continuously provided and liquid effluent is continuously removed. This is typically described as “chemostat” growth or continuous culture. The growth rate as well as the amount of reducing species or oxidizing species may easily controlled,

In this example, a Braun Biostat Model B 2-liter fermentor, associated control box and gas mas flow controllers, was used in conjunction with a 5 feed liquid input (composition described below). Dissolved oxygen and pH was controlled via a B. Braun control box. This apparatus was used to cultivate Methylomonas, which naturally produces Coenzyme Q8.

Media:

| | (M) |
|---|---|
| Feed 1F | |
| H₃PO₄ | 0.048 |
| NH₃ | 0.42 |
| pH | 10 |
| Feed 1G | |
| NaCl | 0.19 |
| CaCl₂:2H₂O | 0.011 |
| pH | 7.0 |
| Feed 1H | |
| MgCl₂:6H₂O | 0.02 |
| KCl | 0.035 |
| pH | 7.0 |
| Feed 2C | |
| H₃BO₃ | 5.11 × 10⁻⁵ |
| ZnSO₄:7H₂O | 1.6 × 10⁻⁴ |
| MnSO₄:H₂O | 4.7 × 10⁻⁵ |
| CoCl₂:6H₂O | 3.2 × 10⁻⁵ |
| Na₂MoO₄:2H₂O | 1.46 × 10⁻⁵ |
| NiCl₂:6H₂O | 1.2 × 10⁻⁴ |
| H₃PO₄ | to pH 6.5 |
| Feed 2D | |
| CuSO₄:5H₂O | 4.1 × 10⁻⁴ |
| FeSO₄:7H₂O | 1.2 × 10⁻³ |
| H₂SO₄ | to pH 1.5 |

These 5 feeds (1F, 1G, 1H, 2C, and 2D) were designed to be pumped simultaneously into the 2-liter culture vessel each at the same volumetric rate. In this experiment, the rate was chosen to be 0.2 mL/minute for each feed for a total rate of 1.0 mL/minute or a culture dilution rate of 60 mL/hr/1400 mL reactor volume which is a dilution rate of 0.043 hr⁻¹. This rate is not critical to the outcome of the experiment but was simply chosen to maximize culture density in order to more easily obtain material for analysis. The culture dilution rate may be as high as 0.33 for this strain before washout of the culture occurs.

Temperature of the fermentation run was approximately 30° C., however the strain grows well over a range from 25° C. to 36-38° C. The pH was set at approximately 6.4. However, this strain grows well over a range from about pH 6.3 to about pH 7.8. Dissolved oxygen is typically kept between 10% and 100% of air saturated water. In this experiment, 20% of air saturation was used to generate the cell mass in the reactor.

Cell biomass in the reactor was analyzed for coenzyme Q8 by harvesting sufficient culture to produce approximately 200 mg of lyophilized biomass. Samples of lyophilized biomass were then stored under nitrogen to prevent further reoxidation prior to analysis. Analysis was performed using the Coenzyme Q8 extraction method described previously under General Methods.

Control of Cellular Redox State in the Reactor to Maximize Reduced Coenzyme Q8 Content of Cells.

Coenzyme Q8 is a redox active molecule known to be a component of many microbial respiratory systems which use oxygen or other electron acceptors as terminal oxidant. Thus, it might be anticipated that when cells are starved for electron donors (i.e. methane and/or methanol), in the presence of excess oxygen, the electron transfer chain components (i.e. Coenzyme Q8) become more oxidized due to lack of reducing power in the cell. Conversely, when cells are starved for oxygen in the presence of excess organic electron donor then electron transfer chain components become more reduced due to the lack of reoxidation by oxygen. The data below (Table 5) shows what happens to the redox state of Coenzyme Q8 when the 1400 mL continuous fermentation of Methylomonas sp. 16a is 1) starved for methane (methane limited) for 1 hour, or 2) is starved for oxygen (oxygen limited) for 1 hour.

TABLE 5
Levels of oxidized and reduced coenzyme Q8 in lyophilized
biomass of Methylomonas

| | CoQ8 reduced | CoQ8 oxidized |
|---|---|---|
| Process | (ppm) | (ppm) |
| Oxygen limited | 1940 | 100 |
| Methane limited | 1300 | 810 |

As shown in Table 5, the simple procedure of limiting oxygen to the culture for 1 hour prior to harvesting sample results in approximately 80% of the coenzyme Q8 being in the reduced form versus 62% when methane is removed for 1 hour. This experiment demonstrates the principal and basic method but does not attempt to optimize the process and it is anticipated that higher levels of reduces coenzyme Q8 may be derived by this process.

What is claimed is:

1. A methanotrophic biomass composition comprising a coenzyme Q8 in a substantially reduced form.

2. The methanotrophic biomass composition of claim 1 wherein the coenzyme Q8 is at a concentration of at least 1000 ppm.

3. The methanotrophic biomass composition of claim 1 comprising bacteria selected from the group genera consisting of Methylomonas, Methylobacter, Methylococcus, and Methylosinus.

4. The methanotrophic biomass composition of claim 3 wherein the bacteria are selected from the group consisting of Methylococcus capsulatus, Methylomonas sp. 16a having the ATCC designation ATCC PTA-2402.

5. A feed composition comprising the methanotrophic biomass according to anyone of claims 1-4.

6. A feed composition according to claim 5 wherein the composition is fish feed.

7. A method for the production of a methanotrophic biomass comprising a coenzyme Q8 in substantially reduced form comprising the steps of:
a) providing a methanotrophic biomass comprising coenzyme Q8 in substantially oxidized form; and
b) processing the methanotrophic biomass of step (a) under anoxic conditions whereby the coenzyme Q8 is reduced.

8. A method for the production of methanotrophic biomass comprising a coenzyme Q8 in substantially reduced form comprising the steps of:
a) providing a methanotrophic bacteria which produces coenzyme Q8 in substantially oxidized form;
b) contacting said methanotrophic bacteria with a growth media comprising:
1) at least one electron donor species; and
2) a source of molecular oxygen;
under substantially oxygen free conditions whereby the coenzyme Q8 is substantially reduced to produce a methanotrophic biomass comprising coenzyme Q8 in substantially reduced form; and
c) optionally isolating the methanotrophic biomass of step b).

9. A method according to claim 8 wherein the at least one electron donor species is selected from the group consisting of methane and methanol.

10. A method according to claim 9 wherein reduced form coenzyme Q8 is produced at a concentration of at least 1000 ppm.

11. A method according to claim 8 wherein isolation of the methanotrophic biomass is conducted under substantially oxygen free conditions.

12. A method according to claim 8 wherein the methanotrophic bacteria is selected from the group consisting of Methylomonas, Methylobacter, Methylococcus, and Methylosinus.

13. A method according to claim 12 wherein the methanotrophic bacteria is Methylomonas or Methylococcus.

14. A method according to claim 13 wherein the methanotrophic bacteria is selected from the group consisting of Methylomonas sp. 16a having ATCC number PTA-2402 and Methylococcus capsulatus having NCIMB number 11132.

15. A method according to claim 12 wherein the methanotrophic bacteria is a high growth methanotrophic bacteria.

16. A method according to claim 7 or claim 8 wherein a reducing agent is added to the methanotrophic biomass.

17. A method according to claim 16 wherein said reducing agent is selected from the group consisting of sugars, pyruvate, sodium hydrosulfite, sodium borohydrite, and ascorbate.

18. A method according to claims 7 or 8 wherein the reduced coenzyme Q8 is isolated, purified, or partially purified from the methanotrophic biomass
